SEQUENCE LISTING (2 sequences)

SEQ ID NO: 1 - protein, 11 aa, Artificial Sequence
  peptide derived from Homo sapiens
KETINEEAAEW

SEQ ID NO: 2 - DNA, 33 bp, Artificial Sequence
  nucleic acide molecule derived from Homo sapiens
aargaracnathaaygargargcngcngartgg

(19) United States
(12) Patent Application Publication
(10) Pub. No.: US 2014/0106345 A1
(43) Pub. Date: Apr. 17, 2014

(54) BIOMARKERS FOR IN VITRO PROGNOSIS AND DIAGNOSIS OF GRAFT AND TRASPLANT REJECTION

(71) Applicant: BIOVIALIFE INC., Toronto (CA)

(72) Inventor: Félix Agbalika, Paris (FR)

(73) Assignee: BIOVIALIFE INC., Toronto, ON (CA)

(21) Appl. No.: 14/122,438

(22) PCT Filed: May 24, 2012
(86) PCT No.: PCT/EP2012/059774

§ 371 Date: Dec. 24, 2013

(30) Foreign Application Priority Data
May 27, 2011 (EP) 11305651.9

(51) Int. Cl. G01N 33/68

(52) U.S. Cl. CPC G01N 33/6893
USPC 435/6.1; 530/327; 536/23.5; 530/387.9; 435/331; 435/7.1; 435/7.92

(57) ABSTRACT

Novel peptides, their derivatives and compositions including the same for their use as a tool in prognosis or diagnosis of a grafted organ distress, notably of graft or transplant rejection.

The present invention relates to novel peptides, their derivatives and compositions containing the same for their use as a tool in prognosis or diagnosis of a grafted organ distress, notably of graft or transplant rejection.

The main function of kidney in living organisms is to clear the blood of waste products or toxic substances. Kidney injury may damage renal structure (glomerular or tubule cell deletion, fibrosis, and renal vasculature alterations) leading to chronic renal excretory dysfunction (Lopez-Novoa et al., J Transl Med 2011, 9: 13). Evolution of chronic kidney diseases can be monitored by key pathological events such as percentage of nephron functionally active, glomerular filtration rate, renal excretory function or by measurement of plasma and urine biomarkers (creatinine, uric acid, . . . ). Progression of chronic kidney diseases has been classified in five stages according to renal dysfunction and renal damage (Levey et al., Ann Intern Med 2003, 139, 137-147). Stage 1 is generally a silent phase in which renal dysfunction can be reversed whereas late stage 4 and stage 5 correspond to renal failure and need for renal replacement therapy such as dialysis or kidney transplant whenever feasible.

Organ transplant can also occurs in other organ systems such as circulatory system (heart), digestive system (liver) and respiratory system (lung). Patients suffering from leukaemia can also receive soft tissue graft, i.e bone marrow graft.

Each year in Europe, more than 15 000 renal grafts, 5 000 liver grafts, 2 000 heart transplants and 1 000 lung grafts are performed. Organ transplantation is often the only treatment for end state organ failure. Cells and tissues, such as haematopoietic stem cells or heart valve are also used for transplantation to restore essential functions. Adverse events and reactions such as graft or transplant rejection often occurred in recipients. This could lead at least to the damage of the graft and eventually to its loss. The use of immunosuppressive drugs helps to improve results of transplantation by modulating immune reaction of recipients. However, these drugs induce a set of adverse events for patients that limit their quality of life or survival expectancies. The most frequent side-effects are nephrotoxicity, cancer and cardiovascular events.

Molecular mechanisms underlying graft or transplant rejection have been identified. Immune rejection is mediated by activation of recipient T-cells against donor cells. Recipient T-cells recognize donor's major histocompatibility complex (MHC) class-I encoded antigens. Infiltration of the transplanted organ by T-cells and other mononuclear leukocytes (such as B-cell and natural killer cells) is responsible for damaging the graft, by activating the complement system and mononuclear cells. Both CD4+ and CD8+ T-cells participate in acute cellular rejection. The production of anti-donor MHC class I and class II antibodies is also associated with acute and chronic graft damage. In chronic graft rejection, the recipient immune response activation is probably not the only cause of rejection. Additional causes such as fibrosis, viral infection or recurrence of the original disease might play an important role in graft rejection (Sanchez-Fueyo et al., Gastroenterology 2011, 140, 51-64).

Rejection episodes are diagnosed by conventional clinical parameters, which are specific of the grafted organ and dependant of genetic factors, and are mostly confirmed by biopsy. At the present time, tissue allograft biopsy with conventional histological examination remains the gold standard for diagnosing rejection among transplanted patients (Racusen et al., The Banff 97 working classification of renal allograft pathology, Kidney International 1999, 55, 713-723). As protocol biopsies are invasive methods, physicians' intervention is needed to collect a small piece of tissue of the grafted organ. Though these tests quite accurately predict graft failure, they usually detect it at a relatively late stage when extensive tissue damage has already occurred. Early detection of graft failure before tissue damage occurrence may help physicians monitoring immunosuppressive drugs more efficiently, minimizing their side effects while prolonging graft survival rates. Identification of proteins in sera indicating which patients are at highest risk of subsequent acute allograft rejection and chronic allograft dysfunction may be helpful in developing new in vitro prognosis and diagnosis assays applicable to a body fluid sample.

The identification of biomarkers for graft and transplant rejection remains challenging. In the past ten years, different strategies have been developed to identify such markers. The first strategy consists in selecting a specific protein for its known implication in the immune reaction system. US 2008/274910 discloses that enhanced expression profile of TIRC-7 (T-cell immune response cDNA 7) in renal grafted patients' blood is determinative for early activation of immune activation which could lead to graft rejection.

More ubiquitous proteins (not involved in immune system) such as TRIB-1, a MAP kinase activator (WO 2007/138011), or lysozyme (JP 2006/6177679) have been identified as markers for in vitro diagnosis and or prognosis of respectively renal or enteric acute graft rejection. In all the previous examples, a single protein was used.

Another strategy was to study the transcriptional profile of a transplanted organ. In WO 2004/074815, the inventors studied expression of known genes that encodes pro-inflammatory or adhesion proteins that mediate immune activation and anti-apoptotic proteins on transplanted renal tissue within 15 minutes after vascular reperfusion. The results demonstrate that delayed graft function, acute rejection or delayed rejection can be predicted from the differential expression of clusters of genes in combination with clinical information.

A more global approach was recently developed in the field of graft rejection. Genomic or proteomic technologies have enabled gene or protein expression profiling of many human diseases.

Microarray tools offer the possibility to detect patterns of differentially expressed gene among hundreds or thousands of genes. In WO 2010/083121, the inventors screened on microarrays platforms the level of expression of whole genome on blood sample of a patient who has received a kidney graft and identify a set of genes which level of expression was confirmed by RT-PCR technology. The gene expression profile is employed to predict the occurrence of an acute rejection response between 6 to 3 months in advance. A similar strategy was developed by using in silico data from biopsy-based gene expression microarray studies from renal and cardiac transplantation (Chen et al., PLoS Computational Biology 2010, 6(9), 1-12). This led to the identification of the three cross-organ acute rejection protein biomarkers i.e, PECAM-1, CXCL-9 and CD44.

In heart transplantation, the Cardiac Allograft Rejection Gene Expression Observation (CARGO) studies led to the development of non-invasive, FDA homologated, commercially available test for acute rejection (www.allomap.com). Measured expression levels of 20 genes are transformed by an algorithm into an integer value that is reported to a score. The clinician uses this score in the overall assessment of the probability of rejection at the time of the testing. This in vitro diagnosis assay can be performed between 2 months to over 6 months post transplanting to assess rejection.

Measurement of protein plasma concentration by using iTRAQ-MALDI-TOF/TOF methodology identified 18 protein group codes with differential relative concentration (Cohen Freue et al., Molecular & Cellular Proteomics 9.9 2010, 1954-1967). Some of these proteins have been previously associated with acute renal graft rejection such as those involved in complement system (MBL2-Mannose Binding protein C), coagulation cascade (SERPINA10 and Factor 11) or inflammatory response (Macrophage-stimulating protein-MSPT-1 and MSPT-9). In this publication, the authors also disclosed an anti-hypertrophic protein secreted by cardiomyocytes called PI16, and a vitamin E-binding member of the albumin family protein called AFM, said proteins having unknown role in renal function. This study strengthens the idea that it is possible to identify biomarkers universally present across all transplanted organs.

Identification of new proteins with unknown role in acute or chronic rejection may provide not only new assays for the prognosis and diagnosis of organ rejection but also new targets for therapeutic intervention. Hence, this could lead to total or partial withdrawal of immunosuppressive drugs or to adapt the regimen or to change the active molecule used to allow graft tolerance in recipient.

Although above mentioned genomic and proteomic technologies constitute powerful tools leading to the identification of new genes or proteins as marker of acute or chronic graft rejection, their implementation remains costly and they are not always available in hospital. Accordingly, there is still a need for alternative markers enabling simple, quick and easy to implement assays for prognosis and diagnosis of graft and transplant rejection.

Thus, one of the aims of the present invention is to provide new proteins being a specific biomarker of a grafted organ distress, notably of graft and transplant rejection.

The present invention allows not only the diagnosis but also the prognosis of graft or transplant rejection in only a few minutes.

The present invention relates to a peptide comprising or consisting of the amino acid sequence SEQ ID NO: 1, or peptide derived from SEQ ID NO: 1, said peptide having at least 65% identity with amino acids 1 to 11 of SEQ ID NO: 1 with the proviso that the peptide is not a native viral protein.

In a preferred embodiment, the peptide of the present invention consists of the amino acid sequence SEQ ID NO: 1, or peptide derived from SEQ ID NO: 1, said peptide having at least 65%, more preferably at least 75%, more preferably at least 85%, more preferably at least 95% identity with amino acids 1 to 11 of SEQ ID NO: 1 with the proviso that the peptide is not a native viral peptide.

By “native viral protein” is meant a proteic sequence which comprises, corresponds to, is present in or is otherwise derived from any proteic sequence which may be found in the genome of a virus. As example of native viral protein, the gag protein from HIV is to be cited.

In a preferred embodiment, the peptide according to the invention has at least 70%, more preferably at least 75%, more preferably at least 80%, more preferably at least 85%, more preferably at least 90% and most preferably at least 95% identity with amino acids 1 to 11 of SEQ ID NO: 1.

By the expression “peptide” is meant a contiguous linear amino acid chain. This contiguous amino acid chain can be either from natural origin or artificial (from chemical synthesis). Methods of chemical synthesis of peptides are well known to one skilled in the art.

Fragments of the peptide of the amino acid sequence SEQ ID NO: 1 are also part of the instant invention. By fragment of peptide is meant at least 5 contiguous amino acid chain selected among the eleven amino acids of peptide of SEQ ID NO: 1 which are able to bind to antibodies raised against peptide of SEQ ID NO: 1.

Variants of the peptide of the amino acid sequence SEQ ID NO: 1 which may be obtained by sequence alterations of said peptide are also part of the instant invention. These alterations can include substitutions, deletions, insertions or mutations. Modified or unusual amino acids can also be enclosed in such variants. Functional groups present on the lateral chains of the amino acid moieties on terminal N or C groups can also be modified according to known methods in the art. Such derivatives include for example esters, aliphatic amides of the carboxyl groups and N-acyl derivates of the free aminogroups. Additionally, amino acids may be subjected to post-translational modifications such as glycosylation, syalylation, acetylation, methylation, amidation, sulfatation, formylation or phosphorylation.

Peptides and variants of the peptide comprising or consisting of the amino acid sequence SEQ ID NO: 1 which may be salted peptides are also part of the instant invention. The salt of the carboxyl group comprises inorganic salts as for example sodium, potassium, calcium salts with organic bases as triethanolamine, arginine or lysine. The salt of the amino group comprises for example salts with inorganic acids as hydrochloric acid or with organic acids as acetic acid.

The peptide of SEQ ID NO: 1 has been called VEA195.

The peptide comprising or consisting of the amino acid sequence SEQ ID NO: 1 is derived from a sera protein of molecular weight of 25-30 kDa, isolated from grafted or transplanted host.

The present invention also relates to an isolated nucleic acid molecule encoding a peptide as defined above, in particular a nucleic acid sequence capable of encoding a peptide comprising or consisting of the amino acid sequence SEQ ID NO: 1 or peptide derived from SEQ ID NO: 1 with the proviso that the peptide is not a native viral peptide, wherein said nucleic acid is SEQ ID NO: 2 or a fragment thereof with the proviso that the nucleic acid molecule is not a native viral nucleic acid.

By “native viral nucleic acid” is meant a nucleic acid sequence which comprises, corresponds to, is present in or is otherwise derived from any nucleic acid sequence which may be found in the genome of a virus. As example of native nucleic acid sequence, the gag gene from HIV is to be cited.

Due to the degeneracy of the genetic code, the codons encoding amino acids of SEQ ID NO: 1 may differ in any of their three positions. For example the amino acid lysine can be specified by AAA or AAG codons (difference in the third position).

By the term “nucleic acid molecule” is meant a single or a double stranded DNA molecule, or a single or double stranded RNA molecule, said RNA molecule being directly deducted from DNA molecule, or a hybrid DNA/RNA molecule, which encodes for the peptide of the present invention.

The present invention also relates to a recombinant nucleic acid construct comprising the above-mentioned nucleic acid molecule operably linked to an expression vector.

By the expression “operably linked” is meant that the aforesaid nucleic acid molecule is linked in a covalent way to an expression vector, permitting ribosome to translate the nucleic acid molecule, or permitting RNA polymerase to produce a mRNA encoding the peptide according to the invention.

By the term “expression vector” is meant a molecule made of nucleic acids, in particular a plasmid, which comprises a promoter region and optionally an enhancer region. The expression vector can include other genes such as an antibiotic resistance gene, in order to select host cells that contain the nucleic acid molecule encoding the peptide according to the invention. Recombinant production techniques that use the aforementioned expression vector are well known to one skilled in the art.

The present invention also relates to a host cell comprising the above-mentioned recombinant nucleic acid construct.

By the term “host cell” is meant any living cell, in particular eukaryotic, but also bacterial cell, or any non-living medium comprising transcriptional and/or translational machinery and optionally organelles from a cell, permitting to amplify the aforesaid nucleic acid construct and/or produce the peptide according to the invention.

The present invention also relates to an antibody or fragment thereof that binds specifically to an epitope, said epitope comprising or consisting of the sequence SEQ ID NO: 1 or of fragments of said peptide.

In a preferred embodiment, the antibody or fragment thereof of the present invention binds specifically to an epitope, said epitope consisting of the sequence SEQ ID NO: 1 or of fragments of said peptide.

By the term “antibody” is meant antibody belonging to any species such as human, mouse, rat, rabbit or goat species. Antibody can also be chimeric antibody, i.e. an antibody which comprises parts originating from different species. Antibody can also be humanised. Antibody can also be antibody fragment which comprise at least one paratope such as Fab, F(ab′)2 or scFv fragments. The antibody of the invention can be a polyclonal or a monoclonal antibody. Monoclonal antibody can also be an anti-idiotypic monoclonal antibody or a Fab fragment of anti-idiotypic antibody. The antibody of the invention can be a natural antibody or a recombinant antibody.

By the term “specifically” is meant that peptide interacts with one antibody without interacting substantially with another antibody which does not structurally resemble the aforesaid antibody.

Methods to generate polyclonal or monoclonal antibodies are well known by the one skilled in the art. Typically, peptide of SEQ ID NO: 1 may be injected with high molecular weight carrier protein to non human mammals (mouse, rabbit, goat or sheep for example) in order to elicit a good immune response. Immunization protocol may be performed several times before the animal sacrifice. Serum of animal is then purified on chromatography column to isolate monoclonal or polyclonal antibody of interest.

Typically, monoclonal antibody results from the selection of a hybridoma secreting an antibody raised against peptide of SEQ ID NO: 1. Such a hybridoma results from the fusion between splenic cells of non human mammal immunized with peptide of SEQ ID NO: 1 and myeloma cells of non human mammal Selection of hybridomas occurs on the bases of their capacity to secrete antibodies able to bind to peptide of SEQ ID NO: 1.

In a preferred embodiment, the antibodies targeting the amino acid sequence SEQ ID NO: 1, or peptide derived from SEQ ID NO: 1 is secreted by the hybridomas deposited at the CNCM (Collection Nationale de Culture de Microorganismes, Institut Pasteur, 25 rue du Docteur Roux, 75724 Paris Cedex 15, France) under the Budapest Treaty on May 4, 2011, under reference numbers CNCM I-4479, CNCM I-4480 and CNCM I-4481 which are part of the present invention.

The present invention also relates to hybridomas 2C7D10E5, 4H7A7G9 and 1H8C9C10C7 deposited at the CNCM on May 4, 2011 under reference numbers respectively CNCM I-4479, CNCM I-4480 and CNCM I-4481.

The peptide according to the present invention is immunogenic, i.e. VEA195 peptide is able to induce a humoral immune response once injected in a mammal. The exact function in graft rejection of the whole protein from which VEA195 peptide has been identified is not yet known. As no one can predict whether this protein has beneficial or deleterious effect on graft rejection, two therapeutic strategies might be developed to avoid graft rejection. If the presence of that protein is needed, the administering to the recipient one or more dose of DNA vaccine before or at the same time performing engraftment with a plasmid comprising a polynucleotide encoding the recipient protein from which VEA195 peptide derived might be helpful. This approach was tested in WO 2009/114735 by using DNA vaccine consisting of a plasmid comprising a polynucleotide encoding the Bax pro-apoptotic protein. In the opposite, if the absence of that protein is needed, depletion of whole protein from which VEA195 peptide derived might be developed by using immobilization antibodies raised specifically against said VEA195 peptide. One of ordinary skills in the art might use a procedure in which blood is taken from the patient's circulation to have a depletion process applied to it before it is returned to the circulation. For example, antibodies raised against VEA195 peptide could be bound to a resin column of an apparatus circulating the blood outside the body. The 25-30 kDa protein from which VEA195 peptide has been identified would be retained on the column whereas whole blood is returned to the body of the grafted patient. The rapid depletion of this protein from blood circulation might limit graft or transplant rejection.

Another aim of the invention is to provide a composition comprising at least one peptide or one antibody as defined above.

Still another aim of the invention is to provide a composition as defined above, for its use as a prognostic, diagnostic or monitoring tool for assessing a grafted organ distress, notably of graft or transplant rejection.

The terms “prognostic” and “diagnostic” relate to respectively determining if a graft or transplant rejection is to occur or is occurring in a mammal

By the term “grafted organ distress” is meant grafted organ dysfunction or grafted organ failure. Loss of organ function may results for example, but is not restricted to, accumulation of substances normally eliminated by the organ or inability for the organ to perform its physiological anabolic or catabolic function. Organ failure may be related to, but is not restricted to, necrosis, fibrosis or inflammatory process.

For example, renal distress can be associated with several damages of grafted kidney such as acute tubular necrosis (ATN), chronic interstitial tubular necrosis (CITN), post transplant lymphoproliferative disease (PTLD), extracapillary glomerulonecrosis or graft rejection which is the highest grade of renal distress.

Grafted Organ distress is not restricted to grafted kidney but might also occur in other grafted or transplanted organ and tissues such as lung, heart, liver and bone marrow.

By the term “graft” is meant introduction of cells or tissue in the body of a recipient mammal that come from another individual of the same species (allograft) or from a different species (xenograft) in order to restore a function altered by a disease or a damage. Hematopoietic cells, cord blood cells, pancreatic beta-cells cells illustrate the most commonly performed cells graft. Skin, bone marrow, part of the liver or corneal grafts are given as examples of tissue graft.

By the term “transplant” is meant introduction of an organ removed from the body of a donor mammal to the body of a recipient mammal whose organ can not exert correctly its function anymore. When organ donor and organ recipient mammals are from the same species it is called allotransplantation. Xenotransplantation occurs when the organ donor and the organ recipient are from two different species. The most currently transplanted organs in mammals are lung, kidney, heart, liver, pancreas and intestine. All the previously mentioned types of graft or transplantation are given to illustrate the field of the Invention but should not be used to restrain the scope of the present invention.

By the expression “graft or transplant rejection” is meant that partial or total destruction of grafted cells, tissue or organ are destroyed by the immune system of the recipient. Three type of organ rejection have been identified: hyperacute, acute and chronic. Hyperacute rejection generally occurs within twenty four hours of the transplantation. Acute rejection occurs within the first six months of the transplantation whereas chronic rejection could appear between six months to several years after transplantation despite the use of immunosuppressive drugs.

The present invention also provides a method for predicting or performing the in vitro prognosis or diagnosis of a grafted organ distress, notably of graft or transplant rejection in a mammal comprising the following steps:
(i) determining the presence or amount of peptide of SEQ ID NO: 1 or of fragments of said peptide,
in a biological sample of said mammal.

In another embodiment, the present invention provides a method for predicting or performing the in vitro prognosis or diagnosis a grafted organ distress, notably of graft or transplant rejection in a mammal comprising the following steps:
(i) determining the presence or amount of antibodies raised against peptide of SEQ ID NO:1 or of fragments of said peptide,
in a biological sample of said mammal.

The term “mammal” includes human as well as other mammals such as e.g pigs, cows, horses, rabbits, cats, dogs. The mammal can be a female or a male, an adult or a child.

According to the invention, detecting or determining the level of peptide of SEQ ID NO: 1 or of fragments of said peptide, or of antibodies raised against peptide of SEQ ID NO: 1 or of fragments of said peptide may be performed by any techniques known in the art and preferably by using an immunoassay method.

By the term “immunoassay” is meant any method wherein the level of peptide of SEQ ID NO: 1 or antibody specifically raised against peptide of SEQ ID NO: 1 peptide is determined using at least one peptide and/or one antibody as defined above in the specification. Immunoassay methods are well known to one of ordinary skill in the art and can have various formats for example in solid or liquid phase, in one or two steps, by a sandwich or a competitive method.

As for example, the colloidal gold immuno-chromatographic assay (CIA) may be used (Cao et al., Am. J. Trop. Med. Hyg. 2007, 76(3), 553-558). This type of immunoassay is particularly easy to implement and qualitative results are obtained in less than 30 minutes, and preferably in less than 20 minutes and more preferably in 10 minutes.

The presence of the antigen in the biological sample may be revealed by a gold colloidal labelled detection antibody, said antibody is able to bind to the peptide or protein of interest, by recognising an epitopic site which is different from that recognised by the capture antibody. Preferably, the capture antibody is bound to solid support, in particular cellulose acetate membrane, and the gold colloidal labelled antibody is considered as the migrating antibody.

As for other examples, ELISA assays, radio-immunoassays or any other detection method may be used to reveal the presence of formed antigen-antibody complexes. Thus, different type of labelling of ligands and/or antibody may be envisaged (radioactive, enzymatic, fluorescent, . . . ).

Measurement determination of peptide of SEQ ID NO: 1 or antibody specifically raised against peptide of SEQ ID NO: 1 peptide is performed on biological sample from a grafted or transplanted recipient a few hours or days after the graft or transplant has occurred. The biological sample can be a body fluid sample or a solid sample such as biopsy of grafted organ or tissue.

By the expression “body fluid sample” is meant any type of sample suitable for use in the methods according to the invention. The biological sample can be selected from the group constituted of a blood sample, a serum sample, a plasma sample, a urine sample or a saliva sample.

The present invention also relates to a kit for the in vitro prognosis or diagnosis of a grafted organ distress, notably of graft or transplant rejection comprising:
(i) at least one antibody suitable for binding an epitope comprising or consisting of peptide of SEQ ID NO:1 or of fragments of said peptide,
(ii) at least one antibody coupled to a detection system suitable for binding the antibody-antigen complex,
and optionally at least one peptide of SEQ ID NO: 1 or of fragments of said peptide.

The present invention also relates to the use of peptide of SEQ ID NO: 1 or of fragments of said peptide as a marker for the in vitro prognosis or diagnosis of a grafted organ distress, notably of graft or transplant rejection in a mammal.

The present invention also relates to the use of a nucleic acid molecule of SEQ ID NO: 2 capable of encoding a peptide of SEQ ID NO: 1 as a marker for the in vitro prognosis or diagnosis of a grafted organ distress, notably of graft or transplant rejection in a mammal.

By the term “marker” is also meant “biomarker”, it relates to any protein and/or nucleic acid molecule which expression modulation is useful for prognosing or diagnosing a graft or transplant rejection.

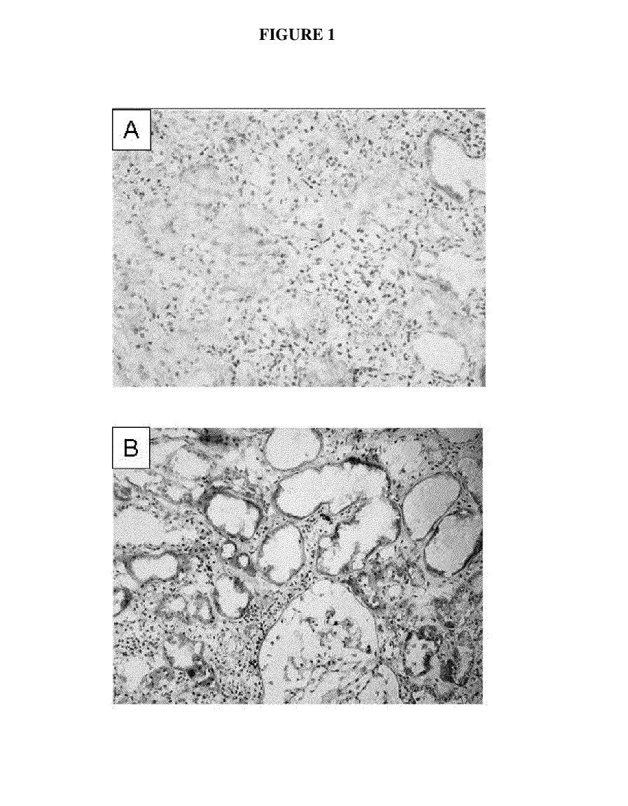

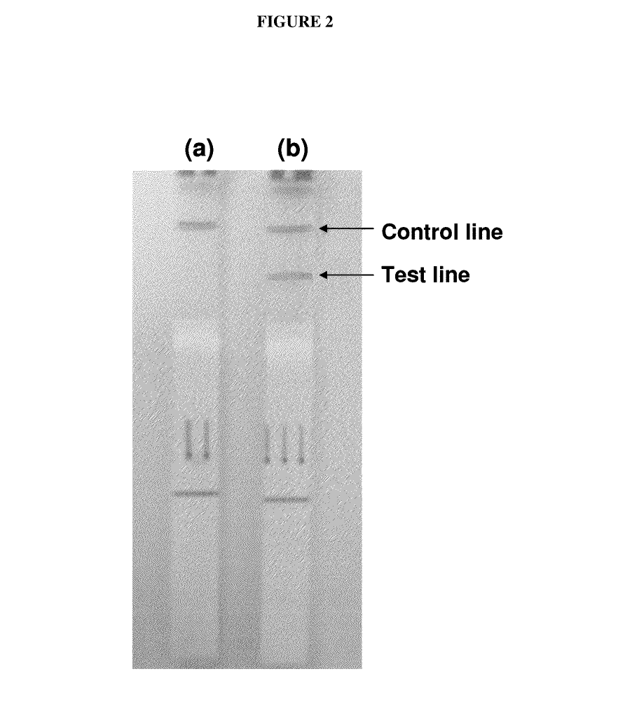

The following examples 1 to 4 and FIGS. 1 to 2 illustrate the invention.

FIG. 1 presents tissular localisation of protein from which VEA195 has been isolated. Renal biopsies were performed on recipients suffering or not from graft rejection according to example 2:

A—Grafted patient not suffering from rejection

B—Grafted patient suffering from rejection

FIG. 2 presents colloidal gold immuno-chromatographic assay (CIA) results. Strips for diagnosis of whole protein from which VEA195 peptide derived in sera of organ recipient according to example 3:

(a)—Grafted patient not suffering from rejection

(b)—Grafted patient suffering from rejection

### EXAMPLE 1

### Isolation, Extraction, Purification of the 25-30 kDa Protein and Synthesis of the VEA195 Peptide Corresponding to the Endogenous Immunogenic Peptide Identified on the 25-30 kDa Protein

### 1.1—Protocol

Five sera samples were collected form renal grafted patient and were cleared of human immunoglobulin (IgG) by protein G sepharose 4 fast flow (Pharmacia, Uppsala, Sweden). A total volume of 15 ml was obtained and loaded onto a Sephadex G-75 (Pharmacia, Uppsala, Sweden) chromatography column for separation by using chloroform methanol 3/1 buffer. Eluates of 200 μl fractions were collected and the analysis of fractions n° 4-5 (400 μl) showed the presence of the 25-30 kDa protein.

The 400 μl were submitted at a dialysis protocol in tubing (Sigma-Aldrich, France) by submersion in 4 L of PBS overnight at 4° C. A final volume of 200 μl of dialysate was finally collected and allowed the identification of a small sequence of 1.2 kDa that has immunogenic properties. The sequencing of this fraction has been defined as VEA195. This peptide has then been synthesized.

VEA195 peptide was assembled on an automated synthesizer by well known method for a person skilled in the art. Briefly, solid phase peptide synthesis was performed using a Boc/Benzyl strategy. Each aminoacid was coupled with a five fold excess, each coupling was performed in DMF in presence of PyBPO, HObt and DIEA. The peptide was cleaved from the resin by treatment with hydrogen fluoride and purified by reverse phase chromatography. The fractions with purity higher than 95% were pooled.

### 1.2—Results

VEA195 peptide was controlled for identity by analytical mass spectrometry and purity by analytical HPLC using a nucleosil C18 250×4.6 mm column. A 10 to 60% gradient of acetonitrile was used for this purification. The electric potential difference of the collected fraction was measured in order to obtain a chromatographic profile. The only peak on the chromatographic profile corresponds to VEA195 peptide that was dried, re-suspended in purified water and then used for immunization of mouse in order to produce specific antibodies.

### EXAMPLE 2

### Immunohistochemistry on Renal Tissues

### 2.1—Protocol

Five-micron sections of formalin-fixed, paraffin-embedded grafted kidney biopsies from grafted patients were cut and mounted on slides Immunohistochemical (IHC) determination of VEA195 previously sequenced from 25-30 kDa protein was performed on an automated staining system (Ventana Medical Systems Tucson, Ariz. USA) according to the manufacturer's recommendations. These slides were deparaffined in xylene, dehydrated through three alcohol passages and transferred to Ventana Kit including solution blocking endogenous peroxidase activity. Anti-VEA195 mouse monoclonal antibodies as 2C7D10E5 (E5), 4H7A7G9 (G9) and 1H8C9C10C7 (C7) were also inserted on the platform and automatically distributed in dilution at 1:100 during the assay. These slides were developed in DAB Map (DAB=di-amino-benzidine) and counterstained with hematoxylin (Ventana) and finally mounted in toluene solution (Eukit, CML, Nemours, France).

### 2.2—Results

They are illustrated in FIG. 1. Biopsy from renal grafted recipient suffering from graft rejection shows a typical 25-30 kDa protein staining both on basal glomerular membrane and on tubules part of the kidney. The cytoplasmic staining in tubular cells is very intense (FIG. 1-B). No 25-30 kDa protein staining appears in the biopsy from healthy (i.e not undergoing graft rejection) renal grafted recipient (FIG. 1-A).

### EXAMPLE 3

### Immuno-Ascendant Chromatography Results

### 3.1—Monoclonal Antibodies Synthesis

About 50 μg of VEA195 peptide diluted in PBS was injected per mouse to a total of 4 mice. The first injection was done intraperitoneally using VEA195 peptide emulsified in complete Freund adjuvant. New injection was performed at day 21 and 42 with test bleeding at day 0, 14, 35 and 56. Upon completion of the last test bleeding an ELISA test was performed. After the last bleed the spleen of the best reacting mouse was operatively taken off. Lymphocytes were isolated and taken up to culture medium.

To keep immunoglobulin-producing lymphocytes alive they were fused with a cell line of mouse myeloma cell line [myeloma SP2/0-Ag14 (ATCC CRL 8287)] in presence of polyethylenglycol. A separation of fused and unfused lymphocytes and myeloma cells was performed on plates filled with HAT medium (Hypoxanthin-Aminopterin-Thymidin). The surviving cells are collected and constitute the hybridoma. The supernatants of the cell cultures are tested on the presence of antigen-specific antibodies and hybridoma cells are frozen to be further grown. Isolation of really monoclonal antibody-producing hybridoma cell line is then performed by diluting the mixture in multiwell plates. By this process one “mother cell” is selected secreting one unique antibody after a cascade of new cell's selection and cloning, ELISA and microscopic controls.

By using this technique, IgG1 or IgG2 kappa isotype monoclonal antibodies were raised against VEA195 peptide: 2C7D10E5 (E5), 4H7A7G9 (G9) and 1H8C9C10C7 (C7).

### 3.2—Immuno Assay Protocol

The double-antibody sandwich technology is used in this assay to enable the detection of whole protein from which VEA195 peptide derived in sera of grafted patients. This test is based on immunochromatography using two specific antibodies raised against VEA195 peptide and a non-specific antibody raised against mouse Ig. The monoclonal E5 capture antibody specifically recognises an epitope of VEA195. Colloidal gold-conjugated G9 migrant antibody recognises a second epitope of VEA195 peptide. Anti-mouse Ig is a control capture antibody that serves as a negative control. The three antibodies were bound linearly to cellulose acetate membrane. The strip is immerged in an assay tube containing the diluted serum of patient in migration buffer (Na₂HPO₄ 50 mM, BSA 1% and NaN₃ 0.10% pH 7.4 from BIOSYNEX). The complex composed of E5 capture antibody-protein from which VEA195 peptide derived-colloidal gold-conjugated G9 migrant antibody forms the test line and the complex composed of Anti-mouse Ig capture antibody-colloidal gold-conjugated G9 migrant antibody forms the control line. The migration of colloidal gold-conjugated antibodies occurs along the test strip. The result was read after 10 minutes. When both the test line and the control line are red, it means that the corresponding sera sample was positive. If only the control line is red, a negative result is indicated.

### 3.3—Results

They are illustrated in FIG. 2. Two lines are present on the strip performed with serum of renal grafted patient suffering from graft rejection (FIG. 2-b): the upper line corresponds to the control line and the lower band is the test line. Only the control line can be seen is the strip test performed with the serum of renal grafted patient not suffering from graft rejection (FIG. 1-a).

A comparative study between colloidal gold immuno-chromatographic assay (CIA) and immunohistochemistry (IHC), performed by hematoxylin and eosin stained section of the grafted organ was done on a total of 20 serum samples of renal grafted patients (table 1, page 17). Final diagnosis was established according to IHC criteria. All experiments were performed with the samples coded and blinded. Analysis by CIA assays was performed retrospectively and blindly, sometimes several months after the diagnosis defined by pathology was held. Results are gathered in Table 1.

TABLE 1
Comparative study between CIA and IHC.

| | | | | | Diagnosis |
|---|---|---|---|---|---|
| Patient | Patient | Date of | IHC | CIA | defined by |
| categories | ID | sampling | analysis | analysis | pathology |
| A1 | 16 | 22 Apr. 2008 | − | − | no rejection |
| | 17 | 27 May 2008 | − | − | |
| | 27 | 26 Jun. 2008 | − | − | |
| A2 | 11 | 05 Feb. 2008 | − | − | acute |
| | 13 | 07 Feb. 2008 | − | − | pyelonephritis - |
| | | | | | no rejection |
| B | 1 | 25 Oct. 2006 | BP | + | AHR |
| | | 13 Feb. 2008 | + | + | |
| | | 27 Jun. 2008 | + | + | |
| | 12 | 18 Jun. 2008 | + | + | |
| C | 14 | 25 Jun. 2008 | BP | + | ACR |
| | 28 | 11 Jun. 2008 | − | + | ND |
| | | 18 Aug. 2008 | + | + | ACR |
| | 34 | 28 Feb. 2006 | BP | ND | ND |
| | | 08 Aug. 2006 | BP | ND | ND |
| | | 28 Jan. 2008 | BP | + | ACR + PTLD |
| | 15 | 02 May 2007 | + | + | ACR + cortical |
| | | | | | necrosis |
| D | 9 | 31 Jan. 2008 | + | + | ATN |
| | 10 | 23 Jan. 2008 | + | + | CITN |
| | 29 | 29 Jan. 2008 | − | + | cryoglobul- |
| | | | | | inemia |
| | 30 | 09 Apr. 2008 | + | + | extracapillary |
| | | | | | glomerulo- |
| | | | | | necrosis |

AHR = acute humoral rejection; ACR = acute cellular rejection; PTLD = post transplant lymphoproliferative disease; ATN = acute tubular necrosis; BP = borderline pathology; CITN = chronic interstitial tubular necrosis; ND = not done.

The comparative study between colloidal gold immuno-chromatographic assay (CIA) and immunohistochemical assay (IHC) was done in function of diagnosis defined by pathology. Patient's profiles are classified as reactive (+), i.e. positive, or unreactive (−), i.e. negative, or borderline pathology (BP), i.e. no histological evidence of graft rejection by IHC. In some cases, since samples were no more available, CIA analysis could not occur and results are indicated as not done (ND).

The 5 serum samples that were found negative with both assays belong to the grafted patient not suffering from graft rejection. This group of patient can be subdivided in two categories: A1 are healthy recipients and A2 are recipients suffering from bacterial infection. Patients belonging to these A1 and A2 categories do not suffer from any renal distress. 13 sera were positive for CIA using anti-VEA195 antibodies. Among those positive samples, 4 were related to acute humoral rejection (AHR) and 4 to acute cellular rejection (ACR). Other forms of renal distress were reactive in 4 serum samples, mainly related to acute tubular necrosis (ATN), chronic interstitial tubular necrosis (CITN) or extracapillary glomerulonecrosis. Patients 1 (first sample), 14 and 34 (third sample) were found borderline pathology (BP, i.e. no histological evidence of graft rejection by IHC) with IHC requiring performing another biopsy. The biopsies performed a few months later confirmed the results obtained previously with the CIA assay. These results clearly demonstrated that anti-VEA195 antibodies are reliable biomarkers for renal distress including graft rejection.

### EXAMPLE 4

### ELISA Assays

### 4.1—Protocol

Microtiter plates are coated with 100 μl of sera from patients diluted in 20 μl of carbonate buffer 50 mM, pH 9.4 overnight at 4° C. Microtiter plates are washed up 3 times with PBS-0.05% Tween 20 buffer. The plates were incubated with 250 μl of PBS buffer and 150 μl of a solution at 5% BSA at room temperature for 2 hours and washed up 3 times with PBS-0.05% Tween 20 buffer. Each well received 100 μl of a 1:4000 dilution E5 antibody in PBS-BSA 1% and the plates are incubated at room temperature for 1 hour. Microtiter plates are washed up 3 times with PBS-0.05% Tween 20 buffer. Peroxydase conjugated goat anti-mouse IgG antibody is added at 1:3000 to each well and incubated for 30 minutes at 37° C. Microtiter plates are washed up 3 times with PBS-0.05% Tween 20 buffer. OPD (Ortho-Phenylenediamine) substrate is then added for 10 minutes. Reaction is stopped by addition of a 4 mM solution of H₂SO₄. Microtiter plates are then read at 415 nm by an optical lecturer.

### 4.2—Results

Results are shown below on table 2, as illustrated on pages 20 and 21. ELISA assay was done with serum samples of 3 blood donors (T) and 8 grafted patients. Means and SD of the optical-density (OD) values obtained for the control sera (blood donors) were used to establish a cut-off value since blood donors are considered as negative samples. Arbitrary method including 3 standard deviations above the mean for negative value was used for the calculation of cut-off value which is 0.068 OD unit in this assay. Values of OD higher than the cut-off value are considered positive whereas values of OD lower than the cut-off value are considered negative. Patients were tested for graft rejection by ELISA assays, retrospectively and blindly, several months after the transplantation occurred and the diagnosis defined by pathology was held.

TABLE 2
anti-VEA195 ELISA assay performance
on grafted recipient serum samples.

| Patient | Patient | Date of | OD values | Diagnosis defined |
|---|---|---|---|---|
| categories | ID | sampling | (415 nm) | by pathology |
| Blood donors | 100 | 01 Mar. 2007 | 0.048 | None |
| | | 01 Apr. 2007 | 0.051 | |
| | | 08 May 2007 | 0.045 | |
| | | 15 May 2007 | 0.050 | |
| | 200 | 20 Jul. 2007 | 0.039 | |
| | | 03 Aug. 2007 | 0.054 | |
| | | 18 Aug. 2007 | 0.061 | |
| | | 30 Aug. 2007 | 0.057 | |
| | | 12 Sep. 2007 | 0.048 | |
| | 300 | 12 Apr. 2008 | 0.048 | |
| | | 30 Apr. 2008 | 0.044 | |
| | | 15 May 2008 | 0.038 | |
| | | 27 May 2008 | 0.047 | |
| A1 | 400 | 10 Jul. 2008 | 0.047 | no rejection |
| | 500 | 15 Aug. 2008 | 0.061 | |
| B | 600 | 20 Apr. 2006 | 0.093 | AHR |
| | | 12 May 2006* | 0.044 | |
| | | 30 May 2006* | 0.056 | |
| | 700 | 30 Apr. 2007 | 0.055 | |
| | | 12 May 2007 | 0.057 | |
| | | 30 May 2007 | 0.084 | |
| | | 24 Jun. 2007 | 0.137 | |
| | | 15 Aug. 2007* | 0.041 | |
| C | 800 | 05 May 2007 | 0.288 | ACR |
| | | 23 Jun. 2007 | 0.135 | |
| | 900 | 08 Feb. 2006 | 2.436 | |
| | | 22 Feb. 2006 | 2.691 | |
| | | 15 Mar. 2006 | 1.281 | |
| | 1000 | 30 Mar. 2005 | 2.377 | |
| | | 06 Apr. 2005 | 2.638 | |
| | | 18 Apr. 2005 | 2.560 | |
| | | 30 Apr. 2005 | 2.557 | |
| | 1100 | 08 May 2005 | 2.797 | |
| | | 03 Apr. 2006 | 0.157 | |
| | | 15 May 2006 | 0.161 | |
| | | 28 May 2006 | 2.661 | |

AHR = acute humoral rejection; ACR = acute cellular rejection

34 serum samples from 8 renal grafted recipients were assayed with anti-VEA195 E5 antibody.

In blood donors (patients ID: 100, 200 and 300), i.e people who are not grafted, the OD value is equal or below 0.068.

In grafted patients who do not suffer from graft rejection several months after transplantations were performed (category A1, patients ID: 400 and 500), the OD values stand also equal or below 0.068. It means that category A1 patients are not suffering from graft rejection. This diagnosis is in concordance with the diagnosis by pathology analysis.

In grafted patients of B category, three samples were analyzed for each patient. The time course of sampling is 40 days for patient 600, and 106 days for patient 700. The first sample of patient 600 shows an OD equal to 0.093 which is higher than the cut-off value, indicating that the 25-30 kDa protein is present in the serum of the patient 600. Concentration of said protein decreases below the limit level of detection of the assay in samples 2 and 3 of said patient. For patient 700, the presence of the 25-30 kDa protein is detected in samples 3 and 4. The 25-30 kDa protein is undetectable in last sample. Patients 600 and 700 were submitted to appropriate treatment (i.e. dialysis, transplant removal, immunosuppressive drugs once the diagnosis of graft rejection was established by pathology. This could explain the decrease of OD values, and consequently the decrease of the 25-30 kDa protein concentration in samples 2 and 3 for patient 600 and in the last sample for patient 700. Those samples are marked with an asterisk in table 2. A modulation of between 1.2 to 2 times the cut-off value seems to be indicative of acute humoral rejection (AHR).

For the four patients of C category, all of the OD values of sample are higher than the cut-off value. The OD value decreases by a half for patients 800 and 900, last sample. For patient 1000, the OD value remains at the same level during the 38 days of the sampling. For patient 1100, the OD value, and consequently the concentration of the 25-30 kDa protein, increases between sample 2 and 3. A modulation of between 2 to 40 times the cut-off value seems to be indicative of acute cellular rejection (ACR).

Finally, it appears that the level of OD value, which reflects the amount of the 25-30 kDa protein in the serum of the patients, is related to the type of graft rejection and may provide diagnostic insights in stages of dysfunction and deterioration of transplantation.

For grafted patients undergoing graft rejection several months after transplantations were performed, the OD values rise from 0.084 to more than 2.7. Consequently, the amount of the 25-30 kDa protein, from which VEA195 peptide derived, in blood sample could be a good tool for prediction of graft rejection. The kinetic expression profile of the 25-30 kDa protein in renal transplant recipients is also probably influenced by the status of the disease and related to the immunosuppressive treatment of patients.

The ELISA assays show that the present invention could be an efficient tool and could provide methods to evaluate grafted organ distress and notably the most deleterious stage illustrated here by graft rejection.

1. A peptide consisting of the amino acid sequence SEQ ID NO: 1, or peptide derived from SEQ ID NO: 1, said peptide having at least 65%, more preferably at least 75%, more preferably at least 85%, more preferably at least 95% identity with amino acids 1 to 11 of SEQ ID NO: 1 with the proviso that the peptide is not a native viral peptide.

2. A nucleic acid molecule capable of encoding a peptide according to claim 1 wherein said nucleic acid is SEQ ID NO: 2 or a fragment thereof with the proviso that the nucleic acid molecule is not a native viral nucleic acid.

3. An antibody or fragment thereof that binds specifically to an epitope consisting of the peptide or of fragments of said peptide according to claim 1.

4. Hybridomas deposited at the CNCM (Paris) under the Budapest Treaty on May 4, 2011, under references numbers CNCM I-4479, CNCM I-4480 and CNCM I-4481, that secrete antibody or fragment thereof as defined in claim 3.

5. A composition comprising at least one peptide according to claim 1 or at least one antibody or fragment thereof that binds specifically to an epitope consisting of the peptide or of fragments of said at least one peptide.

6. A method for assessing a grafted organ distress, notably of graft or transplant rejection in mammal using the composition according to claim 5 as a prognostic, diagonostic or monitoring tool, comprising determining the presences or amount of said composition in a biological sample of said mammal.

7. A method for predicting or performing the in vitro prognosis or diagnosis of a grafted organ distress, notably of graft or transplant rejection in a mammal comprising the following steps:
(i) determining the presence or amount of peptide of SEQ ID NO: 1 or of fragments of said peptide,
in a biological sample of said mammal.

8. A method for predicting or performing the in vitro prognosis or diagnosis of a grafted organ distress, notably of graft or transplant rejection in a mammal comprising the following steps:
(i) determining the presence or amount of antibodies raised against peptide of SEQ ID NO:1 or of fragments of said peptide,
in a biological sample of said mammal.

9. The method according to any claim 7, wherein the level of peptide of SEQ ID NO: 1 or of fragments of said peptide, or of antibodies raised against peptide of SEQ ID NO: 1 or of fragments of said peptide is measured using an immunoassay.

10. The method according to claim 7, wherein the antibody is secreted by one of the hydridomas deposited at the CNCM (Paris) under the Budapest Treaty on May 4, 2011, under references numbers CNCM I-4479, CNCM I-4480 and CNCM I-4481.

11. The method according to claim 7, wherein the biological sample is a body fluid sample.

12. A kit for in vitro prognosis or diagnosis of a grafted organ distress, notably of graft or transplant rejection comprising:
(i) at least one antibody suitable for binding an epitope comprising or consisting of peptide of SEQ ID NO:1 or of fragments of said peptide,
(ii) at least one antibody coupled to a detection system suitable for binding the antibody-antigen complex,
and optionally at least one peptide of SEQ ID NO:1 or of fragments of said peptide.

13. (canceled)

14. A method for predicting or performing the in vitro prognosis or diagnosis of a grafted organ distress, notably of graft or transplant rejection in a mammal, comprising determining the presence or amount of a nucleic acid molecule of SEQ ID NO: 2 capable of encoding a peptide of SEQ ID NO: 1, in a biological sample of said mammal.

15. The method according to claim 8, wherein the level of peptide of SEQ ID NO: 1 or of fragments of said peptide, or of antibodies raised against peptide of SEQ ID NO: 1 or of fragments of said peptide is measured using an immunoassay.

16. The method according to claim 15, wherein the antibody is secreted by one of the hydridomas deposited at the CNCM (Paris) under the Budapest Treaty on May 4, 2011, under references numbers CNCM I-4479, CNCM I-4480 and CNCM I-4481.

17. The method according to claim 8, wherein the antibody is secreted by one of the hydridomas deposited at the CNCM (Paris) under the Budapest Treaty on May 4, 2011, under references numbers CNCM I-4479, CNCM I-4480 and CNCM I-4481.

18. The method according to claim 8, wherein the biological sample is a body fluid sample.
